(19) United States
(12) Patent Application Publication
(10) Pub. No.: US 2004/0236125 A1
(43) Pub. Date: Nov. 25, 2004

(54) Novel structures and compositions comprising sterols and/or stanols and specific classes of anti-inflammatory agents and use thereof in treating or preventing cardiovascular disease, its underlying conditions including hyperlipidemia and other disorders having inflammation as part of their aetiology or presentation

(75) Inventors: James P. Kutney, Vancouver (CA); P. Haydn Pritchard, Vancouver (CA); Yanbing Ding, Vancouver (CA); Kishor M. Wasan, Vancouver (CA)

Correspondence Address: SUSAN M. BEN-OLIEL, BARRISTER & SOLICITOR/PATENT AGENT, 2983 WEST 41ST AVENUE, VANCOUVER, BC, V6N 3C8, CA

(73) Assignee: FORBES MEDI-TECH INC.

(21) Appl. No.: 10/255,558

(22) Filed: Sep. 25, 2002

(51) Int. Cl. C07J 1/00; C07J 9/00

(52) U.S. Cl. 552/508; 552/544

(57) ABSTRACT

The present invention provides, in one aspect, novel derivatives comprising sterols and/or stanols and an NSAID selected from salicylic acids and arylalkanoic acids, including salts of these derivatives, and having one or more of the following formulae:

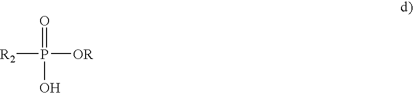

wherein R is a sterol or stanol moiety, R₂ is derived from a salicylic acid or an arylalkanoic acid and n=1-5. Also provided are pharmaceutical compositions comprising one or more of these novel derivatives and methods of treating or preventing cardiovascular disease and its underlying conditions including, without limitation, atherosclerosis, hypercholesterolemia, hyperlipidemia, hypertension, thrombosis, coronary artery disease, and for treating and reducing inflammation including coronary plaque inflammation, bacterial-induced inflammation, viral induced inflammation and inflammation associated with acute pain and surgical procedures which comprises administering to an animal, particularly a human, a non-toxic and therapeutically effective amount of one or more of these compounds or a biologically acceptable salt thereof.

### FIELD OF THE INVENTION

This present invention relates to the field of sterols and stanols and novel derivatives thereof and their use in treating and preventing cardiovascular disease and other disorders.

### BACKGROUND OF THE INVENTION

While recent advances in science and technology are helping to improve quality and add years to human life, the prevention of atherosclerosis, the underlying cause of cardiovascular disease (“CVD”) has not been sufficiently addressed. Atherosclerosis is a degenerative process resulting from an interplay of inherited (genetic) factors and environmental factors such as diet and lifestyle. Research to date suggest that cholesterol may play a role in atherosclerosis by forming atherosclerotic plaques in blood vessels, ultimately cutting off blood supply to the heart muscle or alternatively to the brain or limbs, depending on the location of the plaque in the arterial tree (1,2). Overviews have indicated that a 1% reduction in a person's total serum cholesterol yields a 2% reduction in risk of a coronary artery event (3). Statistically, a 10% decrease in average serum cholesterol (e.g. from 6.0 mmol/L to 5.3 mmol/L) may result in the prevention of 100,000 deaths in the United States annually (4).

Sterols are naturally occurring compounds that perform many critical cellular functions. Phytosterols such as campesterol, stigmasterol and beta-sitosterol in plants, ergosterol in fungi and cholesterol in animals are each primary components of cellular and sub-cellular membranes in their respective cell types. The dietary source of phytosterols in humans comes from plant materials i.e. vegetables and plant oils. The estimated daily phytosterol content in the conventional western-type diet is approximately 60-80 milligrams in contrast to a vegetarian diet which would provide about 500 milligrams per day.

Phytosterols have received a great deal of attention due to their ability to decrease serum cholesterol levels when fed to a number of mammalian species, including humans. While the precise mechanism of action remains largely unknown, the relationship between cholesterol and phytosterols is apparently due in part to the similarities between the respective chemical structures (the differences occurring in the side chains of the molecules). It is assumed that phytosterols displace cholesterol from the micellar phase and thereby reduce its absorption or possibly compete with receptor and/or carrier sites in the cholesterol absorption process.

Over forty years ago, Eli Lilly marketed a sterol preparation from tall oil and later from soybean oil called Cytellin™ which was found to lower serum cholesterol by about 9% according to one report (5). Various subsequent researchers have explored the effects of sitosterol preparations on plasma lipid and lipoprotein concentrations (6) and the effects of sitosterol and campesterol from soybean and tall oil sources on serum cholesterols (7). A composition of phytosterols which has been found to be highly effective in lowering serum cholesterol is disclosed in U.S. Pat. No. 5,770,749 to Kutney et al. and comprises no more than 70% by weight beta-sitosterol, at least 10% by weight campesterol and stigmastanol (beta-sitostanol). It is noted in this patent that there is some form of synergy between the constituent phytosterols, affording even better cholesterol-lowering results than had been previously achieved.

Recently, the role of inflammation in cardiovascular diseases is becoming more understood. Ridker et al. (8) describes a possible role of inflammation in CVD. J. Boyle (9) describes the association of plaque rupture and atherosclerotic inflammation.

Prostaglandins play a major role in the inflammation process and the inhibition of prostaglandin production, especially production of PGG₂, PGH₂ and PGE₂, and have been a common target of anti-inflammatory drug discovery. However, common non-steroidal anti-inflammatory drugs (NSAID's) that are active in reducing the prostaglandin-induced pain and swelling associated with the inflammation process are also active in affecting other prostaglandin-regulated processes not associated with the inflammation process. Thus, use of high doses of most common NSAID's can produce severe side effects, including life-threatening ulcers, that limit their therapeutic potential. An alternative to NSAID's is the use of corticosteroids, which also produce severe adverse effects, especially when long-term therapy is involved.

NSAIDs have been found to prevent the production of prostaglandins by inhibiting enzymes in the human arachidonic acid/prostaglandin pathway, including the enzyme cyclooxygenase (COX). (10). It is now appreciated that there are two isoforms of COX, cyclooxygenase-1 (COX-1) and cyclooxygenase-2 (COX-2) or “prostaglandin G/H synthase IIt. COX-1 is a constitutive isoform found in blood vessels, stomach, and kidney while COX-2 is induced in settings of inflammation by cytokines and inflammatory mediators. The fate of PGG₂/PHG₂ cyclooxygenase products differ from tissue to tissue depending on the particular PGG₂/PHG₂ metabolizing enzymatic activities present. Arachidonic acid also can be converted via 12-lipoxygenase to 12-HPETE and 12-HETE or via the 5-lipoxygenase pathway to a variety of leukotrienes. Aspirin and other NSAIDS inhibit the cyclooxygenase enzyme and prostaglandin production; they do not inhibit lipoxygenase pathways and hence, do not suppress leukotriene production.

It is an object of the present invention to obviate or mitigate the disadvantages of prior known compounds used to treat CVD and underlying disorders including disorders and conditions having inflammation as a part of their aetiology or presentation.

### SUMMARY OF THE INVENTION

The present invention provides, in one aspect, novel derivatives comprising sterols and/or stanols and an NSAID selected from salicylic acids and arylalkanoic acids, including salts of these derivatives, and having one or more of the following formulae:

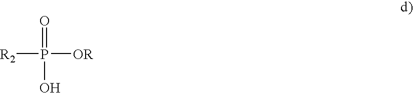

wherein R is a sterol or stanol moiety, R₂ is derived from a salicylic acid or an arylalkanoic acid and n=1-5.

The present invention provides, in another aspect, a composition comprising at least one sterol and/or stanol and at least one an NSAID selected from salicylic acids and arylalkanoic acids.

The present invention also comprises processes of preparing the novel derivatives having the above noted formulae.

The present invention further comprises a pharmaceutical composition for treating or preventing CVD and its underlying conditions including, without limitation, atherosclerosis, hypercholesterolemia, hyperlipidemia, hypertension, thrombosis, coronary artery disease, inflammation including coronary plaque inflammation, bacterial-induced inflammation, viral induced inflammation and inflammation associated with acute pain and surgical procedures which comprises one or more derivatives of sterols and/or stanols and an NSAID selected from salicylic acids and arylalkanoic acids, having one or more of the above noted formulae, and a pharmaceutically acceptable carrier therefor.

The present invention further comprises a pharmaceutical composition for treating or preventing CVD and its underlying conditions including, without limitation, atherosclerosis, hypercholesterolemia, hyperlipidemia, hypertension, thrombosis, coronary artery disease, and for treating and reducing inflammation including coronary plaque inflammation, bacterial-induced inflammation, viral induced inflammation and inflammation associated with acute pain and with surgical procedures which comprises one or more sterols and/or stanols and one or more NSAIDs selected from salicylic acids and arylalkanoic acids, and a pharmaceutically acceptable carrier therefor.

The present invention further provides foods, beverages and nutraceuticals supplemented with derivatives of sterols and/or stanols and an NSAID selected from salicylic acids and arylalkanoic acids, or compositions thereof having one or more of the above noted formulae.

The present invention further provides foods, beverages and nutraceuticals supplemented with a composition comprising one or more sterols and/or stanols and one or more NSAIDs selected from salicylic acids and arylalkanoic acids.

The present invention further provides a method for treating or preventing CVD and its underlying conditions including, without limitation, atherosclerosis, hypercholesterolemia, hyperlipidemia, hypertension, thrombosis, coronary artery disease, and for treating and reducing inflammation including coronary plaque inflammation, bacterial-induced inflammation, viral induced inflammation and inflammation associated with acute pain and surgical procedures which comprises administering to an animal, a non-toxic and therapeutically effective amount of one or more derivatives of sterols and/or stanols and an NSAID selected from salicylic acids and arylalkanoic acids, having one or more of the above noted formulae.

The present invention further provides a method for treating or preventing CVD and its underlying conditions including, without limitation, atherosclerosis, hypercholesterolemia, hyperlipidemia, hypertension, thrombosis, coronary artery disease, inflammation including coronary plaque inflammation, bacterial-induced inflammation, viral induced inflammation and inflammation associated with acute pain and surgical procedures which comprises administering to an animal, a non-toxic and therapeutically effective amount of a composition comprising one or more sterols and/or stanols and one or more NSAIDs selected from salicylic acids and arylalkanoic acids.

It has been found that the derivatives and compositions of the present invention exhibit superior activity in both for treating or preventing CVD and its underlying conditions, particularly hyperlipidemia, and treating conditions having inflammation as a part of their aetiology or presentation. There may be an additive or synergistic therapeutic effect, in both of these respects. Equally importantly, it is believed that when the salicylic acids and/or arylalkanoic acids are either derivatized with the sterol/stanol component as described herein, or merely co-adminstered with sterols/stanols in composition, a lower dosage of the selected NSAID may be required to achieve the desired therapeutic effects. This is important due to the documented long-term adverse effects of the administration of many anti-inflammatory agents such as NSAIDs of which the salicylic acids and arylalkanoic acids form part. These effects and other significant advantages will become apparent herein below.

### BRIEF DESCRIPTION OF THE DRAWINGS

The present invention is illustrated by way the following non-limiting drawings in which:

FIG. 1 is a schematic showing the formation of one derivative of the present invention, a phytostanyl-acetylsalicylate by reaction of an acid chloride with the hydroxyl group of the phytostanol component;

FIG. 2 is a schematic showing the formation of another derivative of the present invention, acetoxyphytostanyl salicylates by reaction of activated phytostanyl chloride with the carboxylate group of the salicylic acid component;

FIG. 3 is a bar graph showing the inhibition of cholesterol absorption by one of the derivatives of the present invention;

FIG. 4 is a bar graph showing percent inhibition of COX-1 in the presence of one of the derivatives of the present invention “FDC-2-4”. SC-560 is an inhibitor of COX-1 with an IC50 of 10 nM. FDC-2-4 is treated with PL/C (PL+) and untreated with PL/C (PL−). “PL only” groups are only treated with PL/C alone (no drug). Data presented as percentage inhibition of mean absorbance compared to 100% activity±standard error, n=3. * indicates p<0.05 vs. 0.45 mM ASA; and

FIG. 5 is a bar graph showing percent inhibition of COX-2 in the presence of one of the derivatives of the present invention “FDC-2-4”. DuP-697 is an inhibitor of COX-2 with an IC50 of 50 nM. FDC-2-4 is treated with PL/C (PL+) and untreated with PL/C (PL−). “PL only” groups are only treated with PL/C alone (no drug). Data presented as percentage inhibition of mean absorbance compared to 100% activity±standard error, n=3. *indicates p<0.05 vs. 0.45 mM ASA

### PREFERRED EMBODIMENTS OF THE INVENTION

The following detailed description is provided to aid those skilled in the art in practising the present invention. However, this detailed description should not be construed so as to unduly limit the scope of the present invention. Modifications and variations to the embodiments discussed herein may be made by those with ordinary skill in the art without departing from the spirit or scope of the present invention.

According to one aspect of the present invention, there are provided novel derivatives of sterol and/or stanol and an NSAID selected from salicylic acids and arylalkanoic acids which are suitable for use per se in treating or preventing CVD and its underlying conditions including, without limitation, atherosclerosis, hypercholesterolemia, hyperlipidemia, hypertension, thrombosis, coronary artery disease, inflammation including coronary plaque inflammation, bacterial-induced inflammation, viral induced inflammation and inflammation associated with acute pain and surgical procedures. The derivatives of the present invention are represented by one or more of the following formulae:

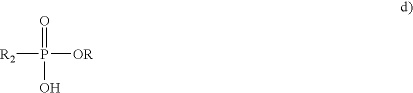

wherein R is a sterol or stanol moiety, R₂ is derived from an NSAID selected from salicylic acids and arylalkanoic acids and n=1-5.

It should be noted that, throughout this disclosure, the terms “derivative”, “structure” and “analogue” and “compound” are used interchangeably to describe this novel unitary group of compounds.

According to another aspect of the present invention, there are provided novel compositions comprising sterols and/or stanols and an NSAID selected from salicylic acids and arylalkanoic acids which are suitable for use per se in treating or preventing CVD and its underlying conditions including, without limitation, atherosclerosis, hypercholesterolemia, hyperlipidemia, hypertension, thrombosis, coronary artery disease, inflammation including coronary plaque inflammation, bacterial-induced inflammation, viral induced inflammation and inflammation associated with acute pain and surgical procedures.

Sterols/Stanols

As used herein, the term “sterol” includes all sterols without limitation, for example: sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, cholesterol and all natural or synthesized forms and derivatives thereof, including isomers. The term “stanol” refers to saturated or hydrogenated sterols including all natural or synthesized forms and derivatives thereof, and isomers. It is to be understood that modifications to the sterols and stanols i.e. to include side chains also falls within the purview of this invention. For example, the purview of this invention clearly includes 24 beta-ethylchlostanol, 24-alpha-ethyl-22-dehydrocholstanol. It is also to be understood that, when in doubt throughout the specification, and unless otherwise specified, the term “sterol” encompasses both sterol and stanol.

The sterols and stanols for use in forming derivatives in accordance with this invention may be procured from a variety of natural sources. For example, they may be obtained from the processing of plant oils (including aquatic plants) such as corn oil and other vegetable oils, wheat germ oil, soy extract, rice extract, rice bran, rapeseed oil, sunflower oil, sesame oil and fish (and other marine-source) oils. They may also be derived from fungi, for example ergosterol, or animals, for example cholesterol. Accordingly, the present invention is not to be limited to any one source of sterols. U.S. Pat. No. 4,420,427 teaches the preparation of sterols from vegetable oil sludge using solvents such as methanol. Alternatively, phytosterols and phytostanols may be obtained from tall oil pitch or soap, by-products of forestry practises as described in U.S. Pat. No. 5,770,749, incorporated herein by reference.

In one preferred form, the derivative of the present invention is formed of naturally-derived or synthesized beta-sitosterol, campestanol, sitostanol, cholesterol or campesterol and each of these derivatives so formed may then be admixed in a composition prior to delivery in various ratios. In another preferred form, the derivative of the present invention is formed with naturally-derived or synthesized sitostanol or with naturally derived or synthesized campestanol or mixtures thereof. The most preferred form of derivative of the present invention comprises either a sitostanyl ester and campestanyl ester or a cholestanyl ester as described further herein.

Salicylic Acids/Arylalkanoic Acids

Suitable anti-inflammatory agents for use within the scope of the present invention are selected from the below-listed specific classes of NSAIDs i.e. those agents which exhibit anti-inflammatory activity in animals, particularly humans, but which do not possess any steroidal structural element. More specifically, the term “arylalkanoic acid” is intended to encompass herein:

arylethanoic (arylacetic) acid compounds such as acemetacin, amfenac sodium, bendazac, glucametacin, oxametacin;

arylpropanoic (arylpropionic) acid compounds such as alminoprofen, ibuprofen, ketoprofen, flurbiprofen, fenoprofen, oxaprozin;

arylbutanoic (arvlbutyric) acid compounds such as bumadizon, butibufen, fenbufen, and xenbucin; and

arylpentanoic (arylvaleric) acid compounds

including all salts thereof.

The term “salicylic acid” as used herein is intended to encompass:

salicylic acid compounds such as acetylsalicylic acid (ASA), aluminium ASA, sodium ASA, ASA glycolates, salicylic acid, salicylic acid glycolates, salicins, salicortin, tremulacin, acetaminosalol; balsalazide, benorylate, gentisic acid, imidazole salicylate, lysine acetylsalicylate, mesalamine, morpholine salicylate, naphthyl salicylate,olsalazine, parsalimide, phenyl salicylate, salicylsulfuric acid, choline magnesium trisalicylate, and other salts, diflunisal, etersalate, fosfosal, salol, salsalate, salacetamide, salicylsalicylic acid, sulfasalazine, olsalazone, including both synthetic and naturally derived forms of all of these compounds;

and including all salts thereof.

Naturally derived salicylates may be extracted, for example, from the bark of Salix alba, S. prupurea L., S. fragilis L. (also known as willow, a deciduous shrub) by techniques which are known and available in the art.

In a most preferred form of the present invention, the NSAID is a salicylic acid derivative, more preferably, ASA or one of its' derivatives.

In one aspect of the present invention, the derivatives are formed between salicylic acid compounds and the sterol/stanol moiety and have one of the following structures:

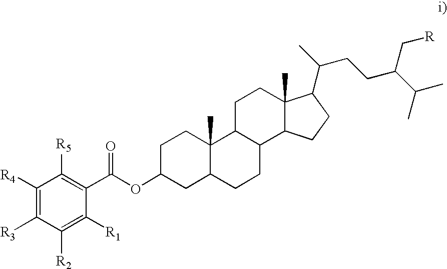

wherein R=H or CH₃ and R1, R2, R3, R4, R5 are selected, independently, from the group consisting of OH, acetyl, halogen (Cl, Br, I, or F) and an alkyl moiety having from 1-5 carbon atoms;

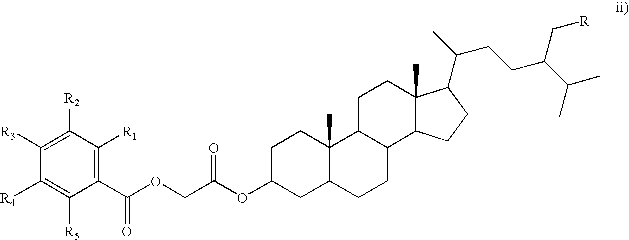

wherein R=H or CH₃ and R1, R2, R3, R4, R5 are independently selected from the group consisting of OH, acetyl, halogen (Cl, Br, I, or F) and an alkyl moiety having from 1-5 carbon atoms;

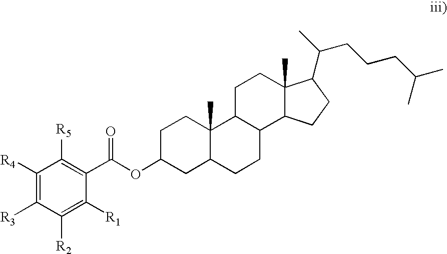

wherein R1, R2, R3, R4, R5 are independently selected from the group consisting of OH, acetyl, halogen (Cl, Br, I, or F) and an alkyl moiety having from 1-5 carbon atoms; and

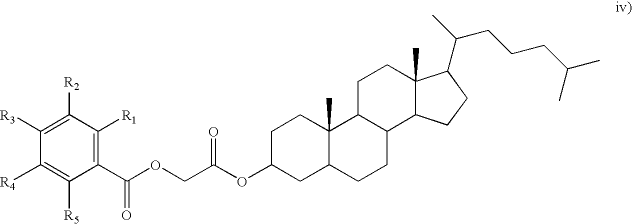

wherein R1, R2, R3, R4, R5 are independently selected from the group consisting of OH, acetyl, halogen (Cl, Br, I, or F) and an alkyl moiety having from 1-5 carbon atoms.

In a most preferred form, the derivative of the present invention is selected from the group consisting of phytostanyl acetylsalicylates, phytostanyl salicylates, acetoxyphytostanyl acetylsalicylates, acetoxyphytostanyl salicylates, acetoxyphytostanyl acetate, cholestanyl salicylates, acetoxycholestanyl salicylates, and acetoxyphytostanyl aminosalicylates as represented by the following formulae:

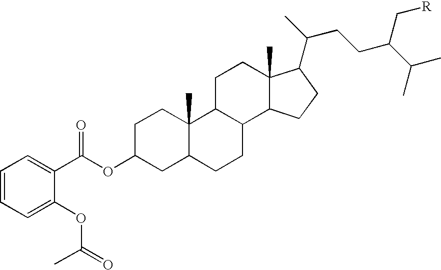

Phytostanyl Acetylsalicylate (wherein R=H or CH₃)

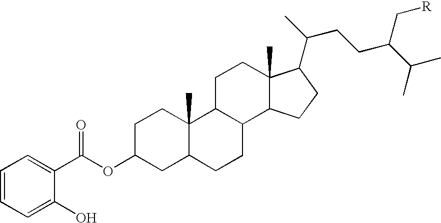

Phyotostanyl Salicylate (wherein R=H or CH₃)

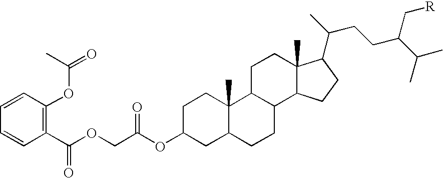

Acetoxyphytostanyl Acetylsalicylate (wherein R=H or CH₃)

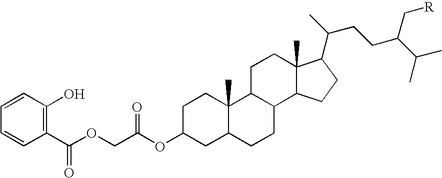

Acetoxyphytostanyl salicylate (wherein R=H or CH₃)

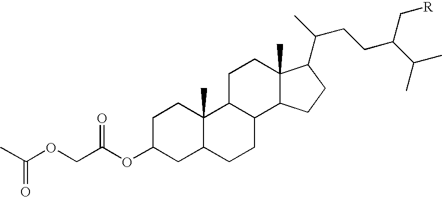

Acetoxyphytostanyl acetate (wherein R═H or CH₃)

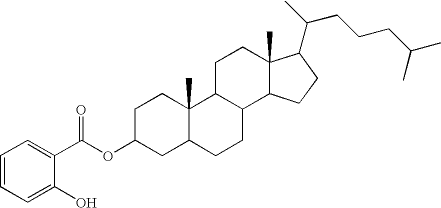

Cholestanyl Salicylate

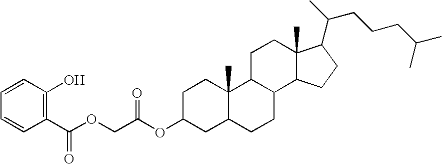

Acetoxycholestanyl salicylate

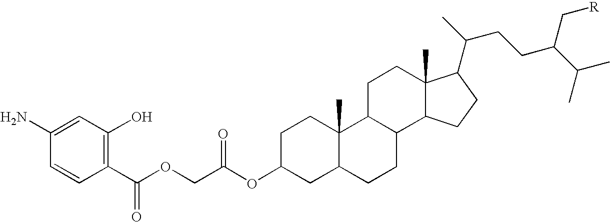

Acetoxyphytostanyl aminosalicylate (wherein R=H or CH₃)

Derivative Formation

a) Ester Formation

There are many processes by which novel structures comprising sterols and/or stanols and the selected anti-inflammatory agent can be formed. In one process, the selected sterol or stanol (or halophosphate, halocarbonate or halo-oxalate derivatives thereof) and the anti-inflammatory agent are mixed together under reaction conditions to permit condensation of the “acid” moiety with the “alcohol” (phytosterol). These conditions are the same as those used in other common esterification reactions in which the acid chloride formed from the acid component and the alcohol component are allowed to react directly or in the presence of a suitable acid catalyst such as mineral acid, sulfuric acid, phosphoric acid, p-toluenesulfonic acid. The organic solvents generally employed in such esterification reactions are ethers such as diethyl ether, tetrahydrofuran, or benzene, toluene or similar aromatic solvents and the temperatures can vary from room to elevated temperatures depending on the reactivity of the reactants undergoing the reaction.

In one preferred embodiment, the process to form the ester derivative comprises “protecting” the hydroxyl groups of the anti-inflammatory or derivatives thereof as esters (for example, as acetate esters) or ethers (for example, methyl ethers) and then condensing the protected anti-inflammatory agent with the reactive sterol/stanol (or its halophosphate, halocarbonate or halo-oxalate) under suitable reaction conditions. In general, such condensation reactions are conducted in an organic solvent such as diethyl ether, tetrahydrofuran, or benzene, toluene or similar aromatic solvents. Depending on the nature and reactivity of the reactants, the reaction temperatures may vary from low (−15° C.) to elevated temperatures.

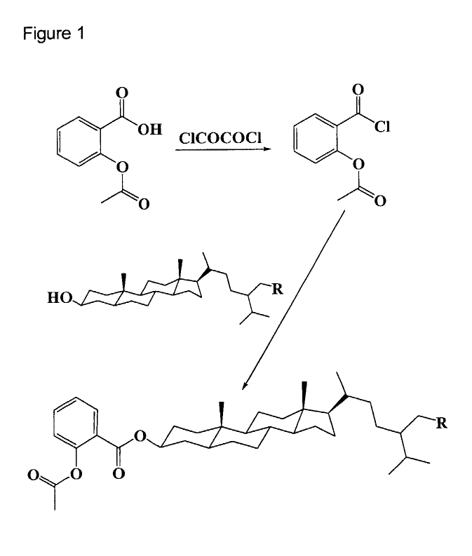

By way of example, FIG. 1 is a schematic showing the formation of the “protected” anti-inflammatory agent (acetylsalicylic acid chloride) and the phytostanol in a condensation reaction yielding one of novel derivatives of the present invention.

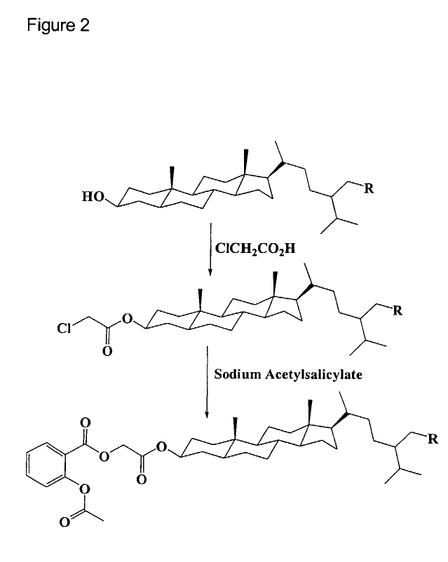

In an alternative, exemplified in the schematic of FIG. 2, the overall formation of the desired ester may be achieved by the creation of an activated chloride in the stanyl or steryl component which is subsequently reacted with the carboxylic acid or carboxylate unit of the anti-inflammatory agent. By this means, there is created a carboxy “linker” between the anti-inflammatory agent and the steryl/stanyl component. This linker may comprise preferably from 1-5 carbon atoms. In Example 2 below, monochloroacetic acid was reacted with a stanol mixture to achieve the stanyl monochloroacetic ester. Similarly, monochloropropionic acid, monochlorobutyric acid and monochlorovaleric acid, or similar acids could be used to lengthen the linker as desired. Although the mechanism of action is unclear, it has been found, in fact, that these derivatives comprising the carboxy linker are the most effective in lipid modulation (cholesterol lowering) and in reducing harmful inflammatory effects.

With respect to the formation of these derivatives, it is to be appreciated that, while selected synthesis processes are described, there are a number of other means by which the variety of derivatives disclosed and claimed can be made. It is well within the purview of a skilled person in this chemical field, once a particular derivative is chosen, to undertake the synthesis using commonly available techniques in the art. For this reason, the complete synthesis of each and every claimed derivative is not described.

Where possible (i.e. where the parent contains a free hydroxyl group), the present invention encompasses the biologically acceptable metal, alkali earth metal, or alkali metal salts of the disclosed derivatives. These salts are generally more water soluble than the corresponding parent compounds and therefore their efficacy and evaluation both in vitro and in vivo is improved.

Salt formation of the derivatives of the present invention can be readily performed by treatment of any parent compound containing a phenolic OH group with a series of bases (for example, sodium methoxide or other metal alkoxides) to produce the corresponding alkali metal salts. Other metal salts of calcium, magnesium, manganese, copper, zinc, and the like can be generated by reacting the parent with suitable metal alkoxides.

Methods of Use

The present invention provides a method for:

1) treating or preventing CVD and its underlying conditions including, without limitation, atherosclerosis, hypercholesterolemia, hyperlipidemia, hypertension, thrombosis, coronary artery disease, and coronary plaque inflammation;

2) treating or ameliorating general inflammation including bacterial-induced inflammation, viral induced inflammation and inflammation associated with acute pain and surgical procedures

which comprises administering to an animal, a non-toxic and therapeutically effective amount of a composition comprising one or more sterols or stanols and at least one NSAID selected from salicylic acids and arylalkanoic acids or one or more derivatives of sterols and/or stanols and an NSAID selected from salicylic acids and arylalkanoic acids, having one or more of the following formulae:

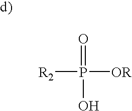

wherein R is a sterol or stanol moiety, R₂ is derived from an NSAID selected from salicylic acids and arylalkanoic acids and n=1-5.

This invention further comprises the use of any of the disclosed compounds for these indications.

The term “therapeutically effective” is intended to qualify the amount of the compound(s) or composition administered in order to achieve one or more of the following goals:

a) treating conditions associated with CVD generally;

b) treating atherosclerosis;

c) treating hypercholesterolemia;

d) treating a hyperlipidic condition;

e) treating hypertension;

f) treating thrombosis;

g) treating coronary artery disease;

h) treating coronary plaque inflammation;

i) treating any inflammatory condition including bacterial or viral-induced inflammation, or inflammation associated with acute pain and surgical procedures; and/or

j) inhibiting COX-1 and/or COX-2 activity

In particular, the compounds of the present invention have been found to be especially useful in addressing at least two significant factors contributing to the multi-factorial presentation of cardiovascular disease: elevated cholesterol levels and inflammation. It is now documented that endothelial inflammatory response, together with plasma cholesterol levels, both play important roles in the development of atherosclerosis (11). Accordingly, it is highly advantageous to administer one compound which simultaneously lowers cholesterol absorption, thereby lowering serum cholesterol and at the same time reduces the inflammation associated with and recognized as part of the disease progression. No other compound to date achieves this beneficial dual effect.

The desired effects described herein may be achieved in a number of different ways. The compounds and compositions of the present invention may be administered by any conventional means available for use in conjunction with pharmaceuticals, nutraceuticals, foods, beverages, and the like.

The amount of the compound or composition which is required to achieve the desired effects will, of course, depend on a number of factors such as the particular compound or composition chosen, the mode of administration and the condition of the patient.

The compounds and compositions of the present invention can be administered to a patient either by themselves, or in pharmaceutical compositions where they are mixed with suitable carriers or excipients.

Use of pharmaceutically acceptable carriers to formulate the compounds and compositions herein disclosed for the practice of the invention into dosages suitable for systemic administration is within the scope of the invention. With proper choice of carrier and suitable manufacturing practice, the compounds and compositions of the present invention, in particular, those formulated as solutions, may be administered parenterally, such as by intravenous injection. The compounds and compositions can be formulated readily using pharmaceutically acceptable carriers well known in the art into dosages suitable for oral administration. Such carriers enable the compounds and compositions of the invention to be formulated as tablets, pills, capsules, liquids, gels, syrups, slurries, suspensions and the like, for oral ingestion by a patient to be treated.

Pharmaceutical compositions, comprising one or more of the compounds of the present invention, include compositions wherein the active ingredients are contained in an effective amount to achieve their intended purpose. Determination of the effective amounts is well within the capability of those skilled in the art, especially in light of the detailed disclosure provided herein.

In addition to the active ingredients these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients include lactose, sucrose, mannitol, sorbitol, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

In another form of the present invention, the compounds and compositions of the present invention may be administered through foods, beverages and nutraceuticals, including, without limitation, the following:

1) Dairy Products—such as cheeses, butter, milk and other dairy beverages, spreads and dairy mixes, ice cream and yoghurt;

2) Fat-Based Products—such as margarines, spreads, mayonnaise, shortenings, cooking and frying oils and dressings;

3) Cereal-Based Products—comprising grains (for example, bread and pastas) whether these goods are cooked, baked or otherwise processed;

4) Confectioneries—such as chocolate, candies, chewing gum, desserts, non-dairy toppings (for example Cool Whip™), sorbets, icings and other fillings;

5) Beverages—whether alcoholic or non-alcoholic and including colas and other soft drinks, juice drinks, dietary supplement and meal replacement drinks such as those sold under the trade-marks Boost™ and Ensure™; and

6) Miscellaneous Products—including eggs and egg products, processed foods such as soups, pre-prepared pasta sauces, pre-formed meals and the like.

The compounds and compositions of the present invention may be incorporated directly and without further modification into the food, nutraceutical or beverage by techniques such as mixing, infusion, injection, blending, dispersing, emulsifying, immersion, spraying and kneading. Alternatively, the compounds and compositions may be applied directly onto a food or into a beverage by the consumer prior to ingestion. These are simple and economical modes of delivery.

### EXAMPLES

The present invention is described by the following non-limiting examples:

### Example 1

### Synthesis of Phytostanyl Acetylsalicylates

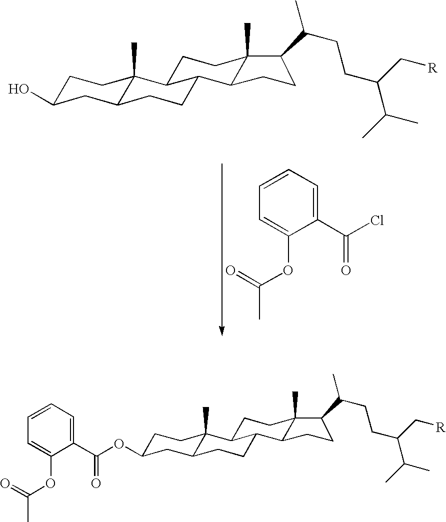

R=Me, H

(i) Synthesis of Acetylsalicylic Acid Chloride

Acetylsalicylic acid (1 g) was suspended in oxalyl chloride (5 ml) and the mixture was refluxed for 1 hr. Excess oxalyl chloride was removed by distillation, and the residue was dried under vacuum overnight to afford a yellowish wax (1.1 g).

(ii) Synthesis of Phytostanyl Acetylsalicylates

To the chloride prepared above, the stanol mixture (2 g, campestanol: 36.4% w/w; sitostanol: 62.3% w/w) and pyridine (10 ml) was added, and the mixture was then stirred overnight at room temperature. The red solution was poured into water (100 ml), filtered, the solid was collected, (2.1 g). The crude product was loaded on a silica gel column (100 ml), eluted with petroleum (30-50° C.):EtOAc (100:3) to yield a white powder, (1.5 g, yield 55%).

### Example 2

### Synthesis of Acetoxyphytostanylacetylsalicylate

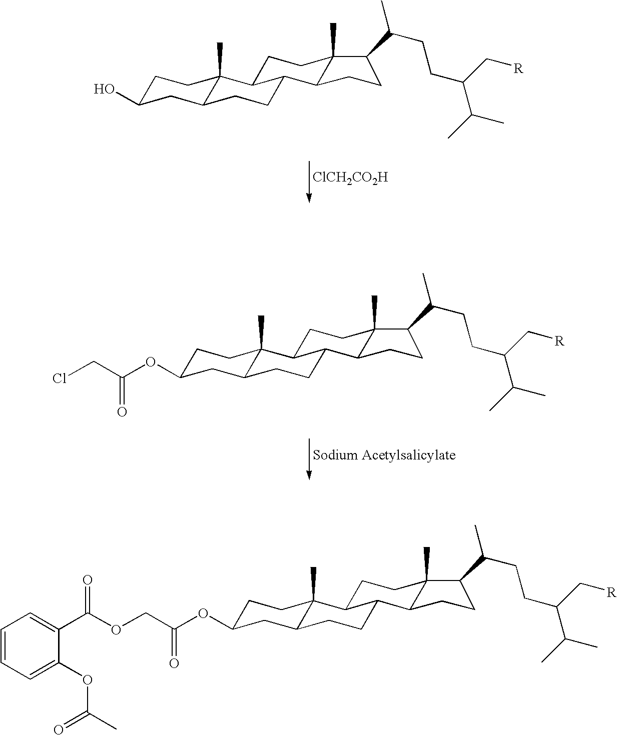

R=Me, H

(i) Synthesis of Phytostanyl Monochloroacetate.

The stanol mixture (4 g, campestanol: 36.4% w/w; sitostanol: 62.3% w/w) in monochloroacetic acid (10 ml) was heated to 120° C. under stirring for 3 hours. After cooling down to room temperature, water (50 ml) was added to the reaction mixture, the precipitate was collected and washed with water (10 ml×2), an dried under vacuum to yield phytostanyl monochloroacetate as a white solid (4.5 g, yield 95%).

¹H NMR (CDCl₃): 4.75 (1 H, m), 4.00 (2H, s).

MS (EI): 492 (M⁺_{Sitostanol ester}), 478 (M⁺_{Campestanol ester}).

(ii) Synthesis of Acetoxyphytostanol Acetylsalicylate

Sodium O-acetylsalicylate (0.5 g) in dry DMF (10 ml) was added to the above prepared phytostanyl monochloroacetate (1.5 g), the mixture was heated to 140° C. with stirring for an hour. After cooling down to room temperature, the mixture was poured into water (100 ml), the off-white solid was collected, dried and weighed (1.8 g, yield 93.4%). The crude material is purified by chromatography on silica gel with an eluting solvent of ethyl acetate hexanes=100:5, to afford a white powder (1.2 g).

¹H NMR (CDCl₃): 8.12 (1H, d), 7.55 (1H, t), 7.30 (1H, t), 7.10 (1H, d), 4.80 (1H, m), 4.75 (2H, s).

¹³C NMR (CDCl₃): 169.58, 167.02, 163.71, 150.85, 134.19, 131.96, 126.02, 123.83, 122.56, 75.29, 61.39.

MS (EI): 636 (M⁺_{Sitostanol ester}), 622 (M⁺_{Campestanol ester}), 594 (M⁺ₛ-15), 580 (M⁺_{c}-15).

IR (cm⁻¹): 2934.5 (C—H), 1764.03 (C═O), 1731.08 (C═O).

### Example 3

### Synthesis of Acetoxycholestanylsalicylate

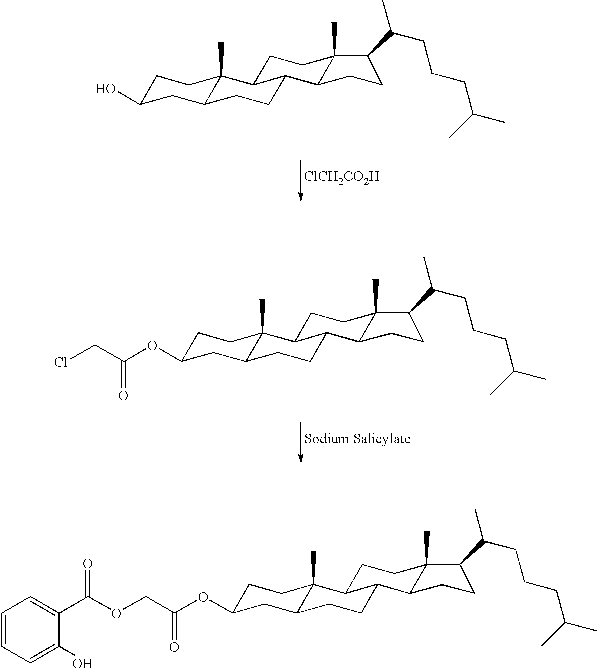

(iii) Synthesis of Cholestanyl Monochloroacetate.

Cholestanol (4 g) in monochloroacetic acid (10 ml) was heated to 120° C. under stirring for 3 hours. After cooling down to room temperature, water (50 ml) was added to the reaction mixture, the precipitate was collected and washed with water (10 ml×2), dried under vacuum, to yield cholestanyl monochloroacetate as a white solid (4.5 g, yield 96%).

¹H NMR (CDCl₃): 4.75 (1H, m), 4.00 (2H, s).

(iv) Synthesis of Acetoxycholestanylacetylsalicylate

Sodium O-acetylsalicylate (0.5 g) in dry DMF (10 ml) was added to the above prepared cholestanyl monochloroacetate (1.5 g), the mixture was heated to 140° C. with stirring for an hour. After cooling down to room temperature, the mixture was poured into water (100 ml), and extracted with hexanes (150 ml), dried over sodium sulfate (20 g), concentrated to remove the solvent, and the waxy solid was collected. After drying, the crude product (2.8 g) was recrystallized in EtOAc:MeOH solvents, to afford a white powder (2.2 g).

¹H NMR (CDCl₃): 10.4 (1H, s), 7.90 (1H, d), 7.50 (1H, t), 7.00 (1H, d), 6.90 (1H, t), 4.80 (1H, m), 4.80 (2H, s).

¹³C NMR (CDCl₃): 169.3, 166.8, 161.71, 136.1, 130.2, 119.3, 126.02, 117.6, 111.8, 75.5, 61.4.

MS (EI): 566 (M⁺).

IR (cm⁻¹): 2930.5 (C—H), 1759 (C═O), 1687.4 (C═O).

### Example 4

### Synthesis of Acetoxyphytostanylsalicylate

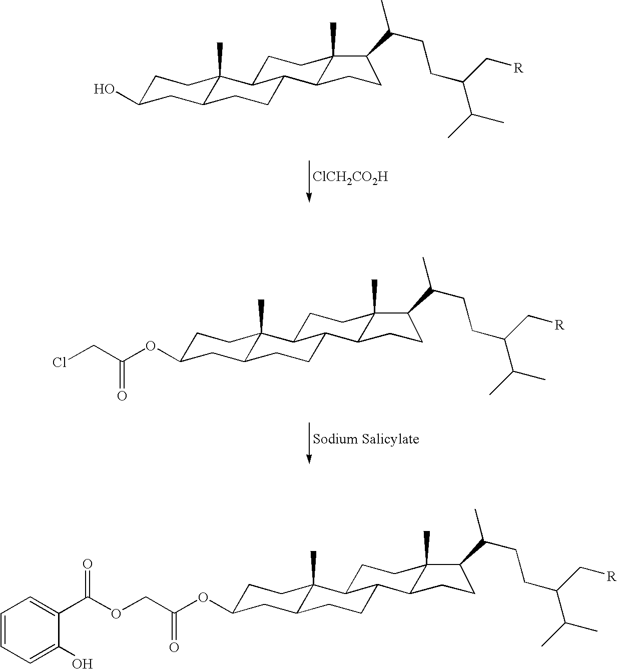

R=H, Me

(v) Synthesis of Acetoxyphytostanylsalicylate

Sodium salicylate (0.5 g) in dry DMF (10 ml) was added to the above prepared phytostanyl monochloroacetate (1.5 g), the mixture was heated to 140° C. with stirring for an hour. After cooling down to room temperature, the mixture was poured into water (100 ml), the off-white solid was collected, dried and weighed (1.8 g, yield 93.4%). After recrystallization in MeOH, a white powder was obtained, (1.5 g).

¹H NMR (CDCl₃): 10.4 (1H, s), 7.90 (1H, d), 7.46 (1H, t), 7.00 (1H, d), 6.90 (1H, t), 4.80 (1H, m), 4.80 (2H, s).

¹³C NMR (CDCl₃): 169.3, 166.8, 161.7, 136.1, 130.3, 131.96, 119.3, 117.6, 111.7, 75.5, 61.4.

MS (EI): 594 (M⁺_{Sitostanol ester}), 580 (M⁺_{Campestanol ester}).

IR (cm⁻¹): 2934.0 (C—H), 1755.9 (C═O), 1680.6 (C═O).

### Example 5

### Synthesis of Acetoxyphytostanylacetate

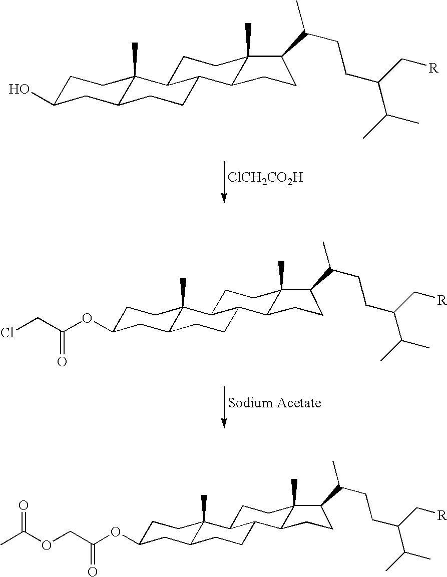

R=Me, H

The procedure was identical to that shown in Example 4 with the exception that sodium acetate was utilized in place of sodium salicylate.

¹H NMR (CDCl₃): 4.90 (1H, m), 4.52 (2H, s), 2.15 (3H, s).

¹³C NMR (CDCl₃): 170.3, 167.3, 75.1, 60.9, 56.4, 56.1, 54.1.

MS (EI): 516 (M⁺_{Sitostanol ester}), 502 (M⁺_{Campestanol ester}).

IR (cm⁻¹): 2933.0 (C—H), 1765.9 (C═O), 1741.8 (C═O).

### Example 6

### Synthesis of Acetoxyphytostanylaminosalicylate

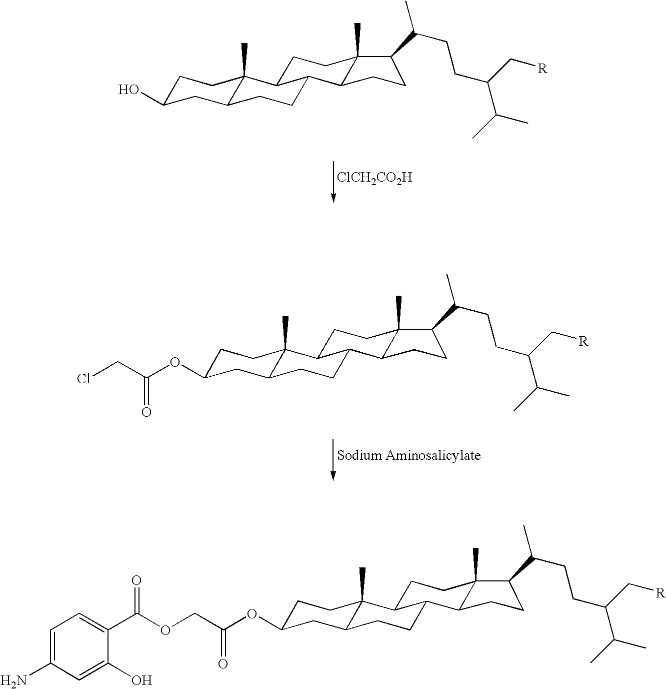

R=H, Me

The procedure was identical to that shown in Example 4 with the exception that sodium 4-amino salicylate was utilized in place of sodium salicylate.

¹H NMR (CDCl₃): 10.6 (1H, s), 7.68 (1H, d), 6.30 (2H, s), 4.78 (1H, m), 4.72 (2H, s), 4.10 (2H, br).

¹³C NMR (CDCl₃): 169.0, 167.3, 163.7, 153.4, 132.0, 107.2, 102.4, 100.9, 75.3, 60.9, 56.4, 56.1, 54.1.

MS (EI): 609 (M⁺_{Sitostanol ester}), 595 (M⁺_{Campestanol ester}).

IR (cm⁻¹): 3377.2 (N—H), 2933.1 (C—H), 1742 (C═O), 1660 (C═O).

### Example 7

### Assays for Cholesterol-Lowering in Rats

Adult male Sprague Dawley rats (˜350 g) were maintained under a 12 h light (0700-1900)/dark cycle and supplied with a laboratory standard diet and water ad libitum prior to starting the experiment.

Following an overnight fasting (12-16 h) rats were divided into 2 groups: control (n=3) and novel compound (acetoxyphytostanyl salicylate which is designated as “FDC-2-4”) group (n=3). Both groups received a single-dose oral gavage at 0700. Blood collection was performed by cardiac puncture 10 hours after oral gavage. Animals were fasted after gavage. Access to water was permitted ad libitum during the experiment. Blood obtained by cardiac puncture was collected in EDTA-coated tubes and centrifuged. Plasma samples were analyzed for [3H] cholesterol by radioactivity.

Adult male Sprague Dawley rats were given one of the derivatives of the present invention in an oral formulation containing:

20 mg/kg sterol/stanol analogue

1 mg unlabeled cholesterol

25 μCi [3H] Cholesterol in an emulsion with Intralipid 10%.

Animals were fasted 12-16 hours before and 10 hours after the oral gavage was performed. Blood samples were collected by cardiac puncture 10 hours following oral gavage. Plasma samples obtained by centrifugation were analyzed for [3H] Cholesterol and results expressed as a percentage of inhibition of cholesterol absorption in the intestine.

Development of a Phytosterol-[3H] Cholesterol Oral Gavage Formulation:

In order to solubilize exogenous radiolabeled cholesterol [25 μCi (approximately 227 ng) a 10% Intralipid emulsion (Kabi Pharmacia) was used.

Intralipid is a sterile non-pyrogenic fat emulsion prepared for administration as a source of calories and essential fatty acids. It is made up of 10% Soybean Oil (refined natural product consisting of a mixture of neutral triglycerides and unsaturated fatty acids), 1.2% egg yolk phospholipids, 2.25% glycerin and water. In addition sodium hydroxide was added to adjust the pH so that the final product pH is 8.0; pH range is 6.0 to 8.9. The major components fatty acids are linoleic (50%), oleic (26%), palmitic (10%), linolenic (9%) and stearic (3.5%). Each animal received 20 mg/kg body weight test substance dissolved in water or other appropriate solvent. The same final volume of the vehicle was administered to rats in the control group.

Cardiac Puncture

Blood sample was collected 10 hours after oral gavage by cardiac puncture and plasma prepared by centrifugation at 40C and 4,000 rpm for 15 minutes. Plasma samples were analyzed for [3H] cholesterol by radioactivity and the effectiveness of novel sterol/stanol compounds reported as a percentage of inhibition of cholesterol absorption compared to controls.

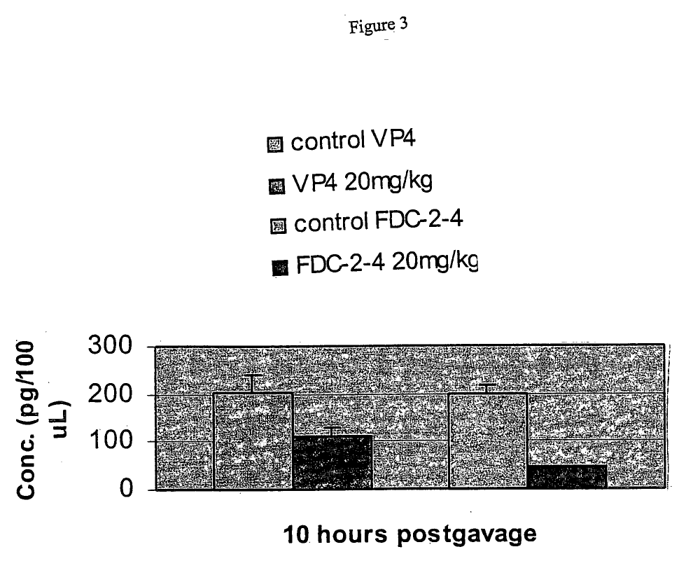

FIG. 3 shows a comparison between the FDC-2-4 and control groups as compared to another effective cholesterol absorption inhibitor (an ascorbate ester) called VP4. The results clearly show that FDC-2-4 significantly outperforms both the controls and VP4. The degree to which FDC-2-4 reduced cholesterol absorption was surprising and unexpected.

### Example 8

### Inhibition of Cyclooxygenase by FDC-2-4

The objective of this study was to determine whether the pancreatic lipase/colipase-treated acetoxyphytostanyl salicylate derivative of the present invention (which is designated as “FDC-2-4”), can inhibit cyclooxygenase (COX) activity.

In summary, FDC-2-4 was treated with a 1:1 ratio of porcine pancreatic lipase and colipase (PL/C). Inhibition of cyclooxygenase was determined using a colorimetric (ovine) COX inhibitor screening assay. The conversion of arachidonic acid to prostaglandin H₂ was monitored by a colorimetric reaction. PL/C-treated FDC-2-4 was constituted in 0, 0.45, 4.5-μM concentrations in a 1 μM hematin/0.1 M Tris-HCl, pH 8.0, buffer. Reaction was initiated by adding 100-μM arachidonic acid and 100-μM tetramethyl-p-phenylenediamine (TMPD—the colourimetric substrate). The optical density of the colored product was determined at 620 nm. Acetylsalicylate was used as a positive control in all experiments.

Pancreatic Lipase/Colipase Treatment:

The enzymatic reaction was carried out in a final volume of 50 mL assay buffer (30 mM Tris-HCl, pH 8.0, 1.0 mM CaCl2, 4 mM taurodeoxycholate), 0.312 mM triolein, in the absence or presence of FDC-2-4. The solution was vortexed for 2 min. and sonicated for 5 min. before adding 2.5 mg porcine pancreatic lipase and 2.5 mg porcine colipase. The reaction was incubated at room temperature for 2 hours.

Inhibition of Cyclooxygenase:

Inhibition was determined using a colorimetric (ovine) COX inhibitor screening assay (Cayman Chemicals). The conversion of arachidonic acid to prostaglandin H2 was monitored by a colorimetric reaction. COX was exposed to the inhibitors for 5 minutes. Reaction was initiated by adding 100-μM arachidonic acid and 100-μM tetramethyl-p-phenylenediamine (TMPD—the colourimetric substrate). The optical density of the colored product was determined at 620 nm 5 minutes after initiation. PL/C-treated FDC-2-4 was constituted in 0, 0.45, 4.5-μM concentrations in a 1 μM hematin/0.1 M Tris-HCl, pH 8.0, buffer. PL/C-treated FDC-2-4 was compared with non-PL/C-treated FDC-2-4 and control (no FDC-2-4 added) in determining COX inhibition. Acetylsalicylate (ASA), was used as a positive control in all experiments. Statistical Analysis on all treatment groups were done using ANOVA and Tukey Posthoc tests.

PL/C-treated FDC-2-4 was compared with non-PL/C-treated FDC-2-4 and control (no added FDC-2-4) in determining COX inhibition. 0.45 μM of PL/C-treated FDC-2-4 inhibited COX-1 activity by 69% compared to non-PL/C-treated FDC-2-4 (20%) and control (5%), n=3. 4.5 μM of PL/C-treated FDC-2-4 inhibited COX-2 by 88% compared to non-PL/C-treated FDC-2-4 (64%) and control (5%), n=3.

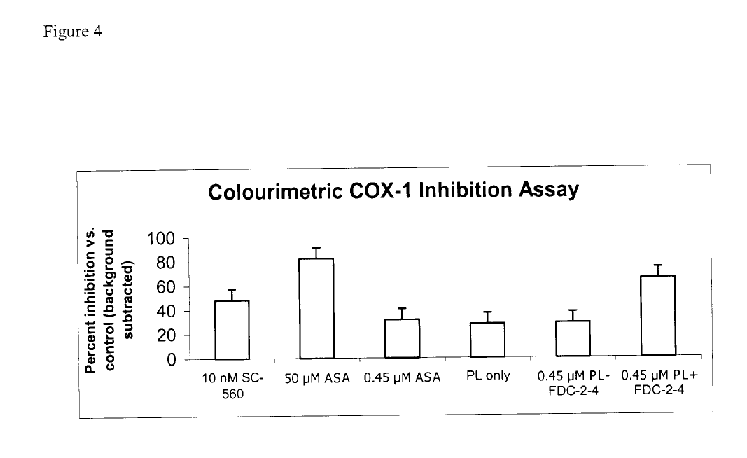

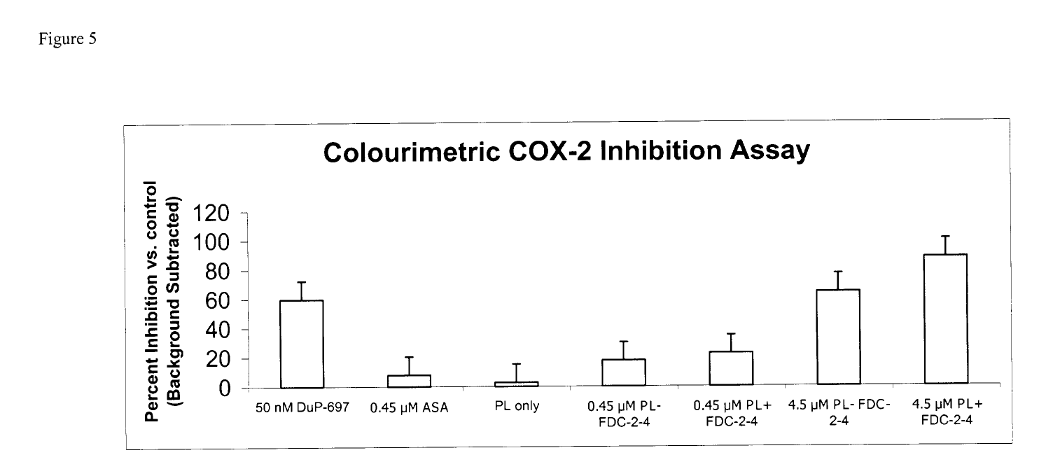

FIGS. 4 and 5 show that FDC-2-4 was found to be effective in inhibiting both isoforms of cyclooxygenase (COX-1 and COX-2). PL/C-treated FDC-2-4 exhibits a stronger inhibition of cyclooxygenase than non-PL/C treated FDC-2-4. These findings suggest that FDC-2-4 is an effective anti-inflammatory agent that is activated by pancreatic lipase/colipase.

Without further elaboration, the foregoing so fully illustrates the present invention that others may, by applying current or future knowledge, adapt the same for use under the various conditions described and claimed herein.

References

1. Law M. R., Wald N. J., Wu., Hacksaw Z A., Bailey A.; Systemic underestimation of association between serum cholesterol concentration and ischemic heart disease in observational studies: Data from BUPA Study; Br. Med. J. 1994; 308:363-366

2. Law M. R., Wald N. J., Thompson S. G.; By how much and how quickly does reduction in serum cholesterol concentration lower risk of ischemic heart disease? Br. Med. J. 1994; 308:367-373

3. La Rosa J. C., Hunninghake D. Bush D. et al.; The cholesterol facts: A summary of the evidence relating to dietary fats, serum cholesterol and coronary heart disease: A joint statement by the American Heart Association and the National Heart, Lung and Blood Institute. Circulation 1990; 81:1721-1733

4. Havel R. J., Rapaport E. Drug Therapy: Management of Primary Hyperlipidemia. New England Journal of Medicine, 1995; 332:1491-1498

5. Kuccodkar et al.; Effects of plant sterols on cholesterol metabolism. Atherosclerosis, 1976; 23:239-248

6. Lees R. S., Lees A. M. Effects of sitosterol therapy on plasma lipid and lipoprotein concentrations. In: Greten H (Ed) Lipoprotein Metabolism. Springer-Verlag, Berlin, Heidelberg, New York, 1976:119-124

7. Lees A. M., Mok H. Y. I., Lees R. S., McCluskey M. A., Grundy S. M. Plant sterols as cholesterol-lowering agents: clinical trials in patients with hypercholesterolemia and studies of sterol balance. Atherosclerosis 1977; 28: 325-338

8. New Eng. J. Med., 336, 973-9 (1999)

9. J. Path. 181, 93-9 (1997)

10. Goodman & Gilman's The Pharmaceutical Basis of Therapeutics, Ninth Edition (1996), McGraw-Hill, page 620.

11. Taubes, G. 2002. Cardiovascular disease: Does inflammation cut to the heart of the matter? Science. 296(5566): 242-245

We claim:

1. A compound comprising a sterol or stanol, including biologically acceptable salts thereof, having one or more of the following formulae: 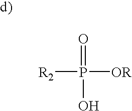
wherein R is a sterol or stanol moiety, R₂ is derived from a salicylic acid or an arylalkanoic acid and n=1-5.

2. The compound of claim 1 wherein the sterol is selected from the group consisting of sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and cholesterol.

3. The compound of claim 1 wherein the stanol is selected from the group consisting of sitostanol, campestanol, stigmastanol, brassicastanol (including dihydrobrassicastanol), desmostanol, chalinostanol, poriferastanol, clionastanol, ergostanol, coprostanol, codistanol, isofucostanol, fucostanol, clerostanol, nervistanol, lathostanol, stellastanol, spinastanol, chondrillastanol, pepostanol, avenastanol, isoavenastanol, fecostanol, pollinastastanol, and cholestanol.

4. The compound of claim 1 wherein the sterol and stanol are in either a natural or synthesized form.

5. The compound of claim 1 wherein the sterol and stanol are in any one of their isomeric forms.

6. The compound of claim 1 wherein the arylalkanoic acid is selected from the group consisting of arylmethanoic (arylformic) acids, arylethanoic (arylacetic) acids, arylpropanoic (arylpropionic) acids, arylbutanoic (arylbutyric) acids and arylpentanoic (arylvaleric) acids.

7. The compound of claim 1 wherein the arylalkanoic acid is selected from the group consisting of acemetacin, amfenac sodium, bendazac, glucametacin, oxametacin, alminoprofen, ibuprofen, ketoprofen, flurbiprofen, fenoprofen, oxaprozin, bumadizon, butibufen, fenbufen, and xenbucin.

8. The compound of claim 1 wherein the salicylic acid is selected from the group consisting of acetylsalicylic acid (ASA), aluminium ASA, sodium ASA, ASA glycolate, salicylic acid, salicylic acid glycolates, salicins, salicortin, tremulacin, choline magnesium trisalicylate, diflunisal, etersalate, fosfosal, salol, salsalate, salacetamide, salicylsalicylic acid, sulfasalazine, and olsalazone.

9. The compound of claim 1 having the following formula: 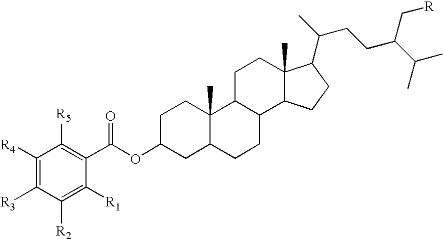
wherein R is selected from H and CH₃ and R1, R2, R3, R4, R5 are selected, independently, from the group consisting of OH, acetyl, halogen (Cl, Br, I, or F) and an alkyl moiety having from 1-5 carbon atoms.

10. The compound of claim 1 having the following formula: 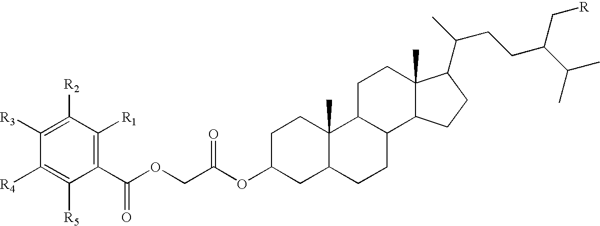
wherein R is selected from H and CH₃ and R1, R2, R3, R4, R5 are independently selected from the group consisting of OH, acetyl, halogen (Cl, Br, I, or F) and an alkyl moiety having from 1-5 carbon atoms;

11. The compound of claim 1 having the following formula: 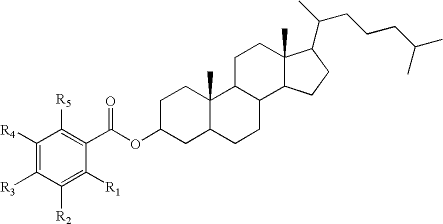
wherein R1, R2, R3, R4, R5 are independently selected from the group consisting of OH, acetyl, halogen (Cl, Br, I, or F) and an alkyl moiety having from 1-5 carbon atoms.

12. The compound of claim 1 having the following formula: 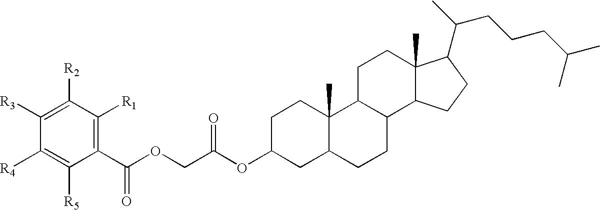
wherein R1, R2, R3, R4, R5 are independently selected from the group consisting of OH, acetyl, halogen (Cl, Br, I, or F) and an alkyl moiety having from 1-5 carbon atoms.

13. The compound of claim 1 selected from the group consisting of phytostanyl acetylsalicylates, phytostanyl salicylates, acetoxyphytostanyl acetylsalicylates, acetoxyphytostanyl salicylates, acetoxyphytostanyl acetate, cholestanyl salicylates, acetoxycholestanyl salicylates, and acetoxyphytostanyl aminosalicylates.

14. The compound of claim 1 having the formula: 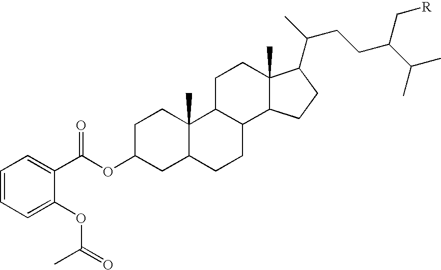
wherein R is selected from H and CH₃.

15. The compound of claim 1 having the formula: 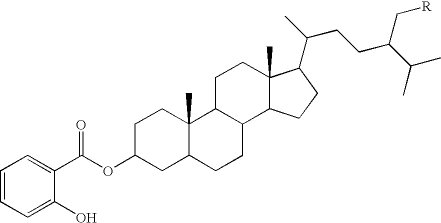
wherein R is selected from H and CH₃.

16. The compound of claim 1 having the formula: 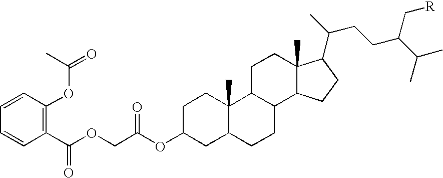
wherein R is selected from H and CH₃.

17. The compound of claim 1 having the formula: 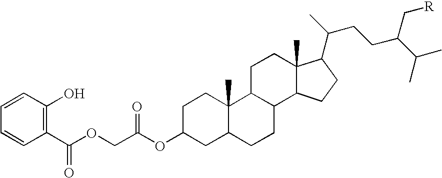
wherein R is selected from H and CH₃.

18. The compound of claim 1 having the formula: 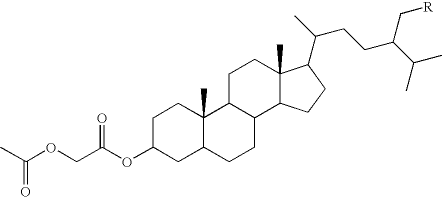
wherein R is selected from H and CH₃.

19. The compound of claim 1 having the formula:

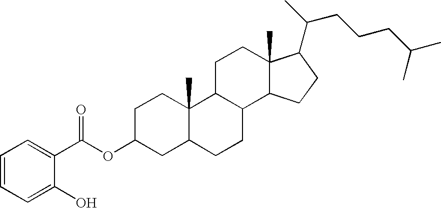

20. The compound of claim 1 having the formula:

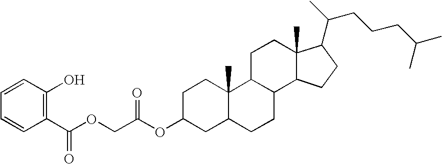

21. The compound of claim 1 having the formula:

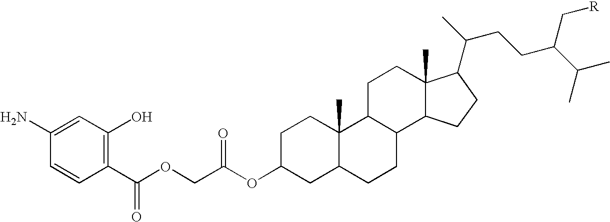

22. A pharmaceutical composition for treating or preventing cardiovascular disease and its underlying conditions including, without limitation, atherosclerosis, hypercholesterolemia, hyperlipidemia, hypertension, thrombosis, coronary artery disease, and for treating inflammation including coronary plaque inflammation, bacterial-induced inflammation, viral induced inflammation and inflammation associated with acute pain and surgical procedures said composition comprising at least one compound having one or more of the following formulae: 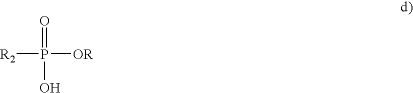
wherein R is a sterol or stanol moiety, R₂ is derived from a salicylic acid or an arylalkanoic acid and n=1-5, including all biologically acceptable salts thereof, and a pharmaceutically acceptable carrier therefor.

23. The composition of claim 22 wherein the arylalkanoic acid is selected from the group consisting of arylmethanoic (arylformic) acids, arylethanoic (arylacetic) acids, arylpropanoic (arylpropionic) acids, arylbutanoic (arylbutyric) acids and arylpentanoic (arylvaleric) acids.

24. The composition of claim 22 wherein the arylalkanoic acid is selected from the group consisting of acemetacin, amfenac sodium, bendazac, glucametacin, oxametacin, alminoprofen, ibuprofen, ketoprofen, flurbiprofen, fenoprofen, oxaprozin, bumadizon, butibufen, fenbufen, and xenbucin.

25. The composition of claim 22 wherein the salicylic acid is selected from the group consisting of acetylsalicylic acid (ASA), aluminium ASA, sodium ASA, ASA glycolate, salicylic acid, salicylic acid glycolates, salicins, salicortin, tremulacin choline magnesium trisalicylate, diflunisal, etersalate, fosfosal, salol, salsalate, salacetamide, salicylsalicylic acid, sulfasalazine, and olsalazone.

26. The composition of claim 22 wherein the compound has the following formula: 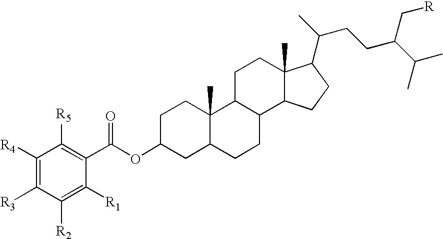
wherein R is selected from H and CH₃ and R1, R2, R3, R4, R5 are selected, independently, from the group consisting of OH, acetyl, halogen (Cl, Br, I, or F) and an alkyl moiety having from 1-5 carbon atoms.

27. The composition of claim 22 wherein the compound has the following formula: 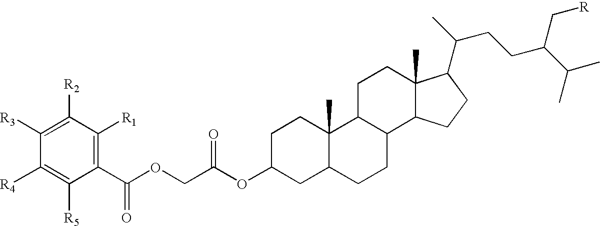
wherein R is selected from H and CH₃ and R1, R2, R3, R4, R5 are independently selected from the group consisting of OH, acetyl, halogen (Cl, Br, I, or F) and an alkyl moiety having from 1-5 carbon atoms.

28. The composition of claim 22 wherein the compound has the following formula: 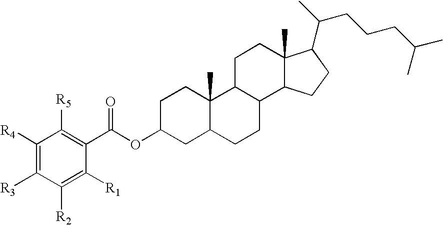
wherein R1, R2, R3, R4, R5 are independently selected from the group consisting of OH, acetyl, halogen (Cl, Br, I, or F) and an alkyl moiety having from 1-5 carbon atoms.

29. The composition of claim 22 wherein the compound has the following formula: 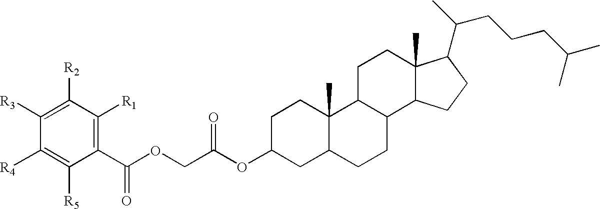
wherein R1, R2, R3, R4, R5 are independently selected from the group consisting of OH, acetyl, halogen (Cl, Br, I, or F) and an alkyl moiety having from 1-5 carbon atoms.

30. The composition of claim 22 wherein the compound is selected from the group consisting of phytostanyl acetylsalicylates, phytostanyl salicylates, acetoxyphytostanyl acetylsalicylates, acetoxyphytostanyl salicylates, acetoxyphytostanyl acetate, cholestanyl salicylates, acetoxycholestanyl salicylates, and acetoxyphytostanyl aminosalicylates.

31. A method for treating or preventing cardiovascular disease and its underlying conditions including, without limitation, atherosclerosis, hypercholesterolemia, hyperlipidemia, hypertension, thrombosis, coronary artery disease, and for treating and reducing inflammation including coronary plaque inflammation, bacterial-induced inflammation, viral induced inflammation and inflammation associated with acute pain and surgical procedures which comprises administering to an animal, a non-toxic and therapeutically effective amount of one or more compounds having the following formulae: 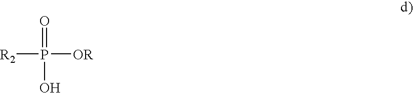
wherein R is a sterol or stanol moiety, R₂ is derived from a salicylic acid or an arylalkanoic acid and n=1-5, or any biologically acceptable salt thereof.

32. The method of claim 31 wherein arylalkanoic acid is selected from the group consisting of arylmethanoic (arylformic) acids, arylethanoic (arylacetic) acids, arylpropanoic (arylpropionic) acids, arylbutanoic (arylbutyric) acids and arylpentanoic (arylvaleric) acids.

33. The method of claim 31 wherein the arylalkanoic acid is selected from the group consisting of acemetacin, amfenac sodium, bendazac, glucametacin, oxametacin, alminoprofen, ibuprofen, ketoprofen, flurbiprofen, fenoprofen, oxaprozin, bumadizon, butibufen, fenbufen, and xenbucin.

34. The method of claim 31 wherein the salicylic acid is selected from the group consisting of acetylsalicylic acid (ASA), aluminium ASA, sodium ASA, ASA glycolate, salicylic acid, salicylic acid glycolates, salicins, salicortin, tremulacin, choline magnesium trisalicylate, diflunisal, etersalate, fosfosal, salol, salsalate, salacetamide, salicylsalicylic acid, sulfasalazine, and olsalazone.

35. The method of claim 31 wherein the compound has the formula: 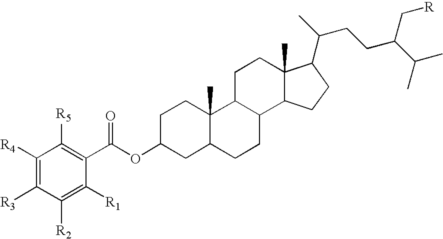
and wherein R is selected from H and CH₃ and R1, R2, R3, R4, R5 are selected, independently, from the group consisting of OH, acetyl, halogen (Cl, Br, I, or F) and an alkyl moiety having from 1-5 carbon atoms.

36. The method of claim 31 wherein the compound has the formula: 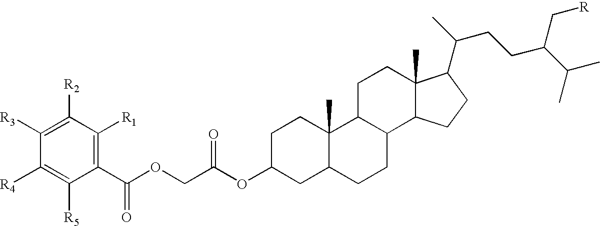
and wherein R is selected from H and CH₃ and R1, R2, R3, R4, R5 are independently selected from the group consisting of OH, acetyl, halogen (Cl, Br, I, or F) and an alkyl moiety having from 1-5 carbon atoms;

37. The method of claim 31 wherein the compound has the formula: 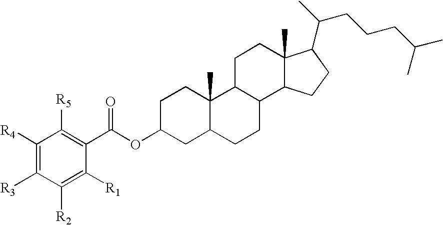
and wherein R1, R2, R3, R4, R5 are independently selected from the group consisting of OH, acetyl, halogen (Cl, Br, I, or F) and an alkyl moiety having from 1-5 carbon atoms.

38. The method of claim 31 wherein the compound has the formula: 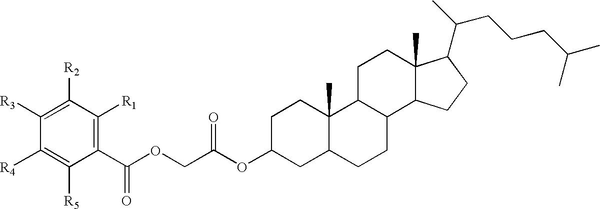
wherein R1, R2, R3, R4, R5 are independently selected from the group consisting of OH, acetyl, halogen (Cl, Br, I, or F) and an alkyl moiety having from 1-5 carbon atoms.
